(19) United States
(12) Patent Application Publication
(10) Pub. No.: US 2011/0002927 A1
(43) Pub. Date: Jan. 6, 2011

(54) ANTIGEN-BINDING POLYPEPTIDES AGAINST CARTILAGE DEGENERATION

(75) Inventors: David M Urech, Hombrechtikon (CH); Peter Lichtlen, Gattikon (CH)

Correspondence Address: MARSHALL, GERSTEIN & BORUN LLP, 233 SOUTH WACKER DRIVE, 6300 WILLIS TOWER, CHICAGO, IL, 60606-6357, US

(73) Assignee: Delenex Therapeutics AG, Schlieren (CH)

(21) Appl. No.: 12/865,365

(22) PCT Filed: Feb. 5, 2009
(86) PCT No.: PCT/CH09/00045

§ 371 Date: Sep. 20, 2010

Related U.S. Application Data
(60) Provisional application No. 61/026,317, filed on Feb. 5, 2008.
(60) Provisional application No. 61/088,876, filed on Aug. 14, 2008.

(51) Int. Cl. A61K 39/395; C07K 16/18; C07K 16/28; C07K 16/24; C07H 21/00; C12N 15/63; C12N 5/10; C12P 21/02; A61P 19/04; A61P 19/02

(52) U.S. Cl. 424/135.1; 530/387.3; 536/23.53; 435/320.1; 435/328; 435/69.6

(57) ABSTRACT

The invention provides an antigen-binding polypeptide which is able to penetrate into the cartilage. The disclosed polypeptide, compositions and methods are suitable for the treatment, prevention and/or delay of progression of cartilage degeneration.

### TECHNICAL FIELD

The present invention relates to pharmaceutical agents against cartilage degeneration.

### BACKGROUND ART

Articular cartilage is composed of chondrocytes embedded in an extracellular matrix. Said matrix is mainly composed of water and further comprises type II collagen and aggrecan, a cartilage-specific proteoglycan. The collagen portion confers tensile strength to the cartilage, whereas the proteoglycan portion absorbs water and thereby provides the ability to resist compression and distribute load.

Cartilage degeneration is observed in a number of conditions, among them osteoarthrits (OA). The degeneration is driven by a multitude of cytokines, growth factors and proteases. Initially, degeneration can be observed at the articular surface in form of fibrillation, leading to the appearance of fissures. Later on, progressive loss of cartilage thickness is observed, resulting from the over-catabolism of the proteoglycan-hyaluronate complex. The degeneration is catalyzed by metalloproteinases such as glycosidases and hexosaminidases (se e.g. US2007197471). Finally, the collagen network is attacked. A positive feedback loop may be observed, in which the degradation products of the matrix molecules stimulate degradation. Cellular responses to the mentioned feedback loop involve the production of cytokines such as IL-1 and TNFalpha which are known to induce expression of matrix metalloproteases (see Goldring 2000, Arthritis & Rheumatism Vol 43 pp 1916-1926; Kobayashi, M. et al. (2005): Role of Interleukin-1 and Tumor Necrosis Factor alpha in matrix degradation of human osteoarthritic cartilage. Arthritis & Rheumatism, Vol. 52(1), pp. 128-135; and Gerwin, N. et al. (2006), Adv. Drug Delivery Rev. 58, pp. 226-242).

Kobayashi, M. et al. (2005) showed in vitro that the inhibition of IL-1 and/or TNFalpha arrested the degradation of type II collagen and proteoglycan in OA cartilage (see Kobayashi, M. et al. (2005): Role of Interleukin-1 and Tumor Necrosis Factor alpha in matrix degradation of human osteoarthritic cartilage. Arthritis & Rheumatism, Vol. 52(1), pp. 128-135). It is therefore considered that regulation of degradation and degeneration of the extracellular matrix leads to treatment of joint diseases (see EP1547617).

OA, also known as degenerative arthritis, is the most common type of arthritis and the leading cause of disability in Europe, the USA and Japan, with an estimated prevalence of 36-48% of the population. Because of the growing proportion of elderly people and the increasing incidence of other risk factors for OA (e.g. obesity and inactive life style), said number is expected to grow (Gerwin, N. at al. (2006), Adv. Drug Delivery Rev. 58, pp. 226-242). Despite the increasing need for effective OA treatment, current therapies only treat signs and symptoms, i.e. pain alleviation, but not the underlying structural changes of the articular cartilage. The current therapies involve the administration of simple analgesics, non-steroidal anti-inflammatory drugs or intraarticular injected glucocorticoids and hyaluronic acid formulations. Hence, there is a largely unmet medical need for the treatment of OA.

As a complication to the treatment of cartilage degeneration, articular cartilage is avascular and alymphatic; as a result, molecules, such as nutrients or pharmaceuticals, must be able to diffuse from the synovial fluid through the dense cartilage matrix to reach the chondrocytes (Gerwin, N. et al. (2006), Adv. Drug Delivery Rev. 58, pp. 226-242). The permeability of solutes, in particular of large molecules through cartilage, among them IgG antibodies, has been studied (Maroudas A., (1976), J. Anat. 122(2), pp. 335-347). The partition coefficients were found to decrease very steeply with increase in size of the solutes; consequently, the passage of larger molecules is limited by the pore size of the matrix meshwork, and, therefore, strongly dependent on the local concentration of glycosaminglycans. Additional work on the penetration of and the persistence in articular cartilage of proteins of various sizes and various pI's has been done by van Lent and colleagues. They found that the penetration of and the persistence within articular cartilage of cationic proteins is much more pronounced when compared to anionic proteins (van Lent, P. L. E. M. et al. (1987), J. Rheumatol. 14(4), pp. 798-805). Mouse in vivo studies performed by vant Lent et al. confirmed his earlier findings in large parts (van Lent, P. L. E. M. et al. (1989), J. Rheumatol. 16(10), pp. 1295-1303). He found that cartilage penetration is better the smaller and the more cationic (high pI) a particular protein is. Cartilage retention, on the other hand, was most pronounced for large cationic proteins, indicating that these proteins effectively bind to the negatively charged cartilage component glycosaminoglycan (GAG), whereas cartilage binding by small cationic proteins is much less efficient. The upper range for cartilage penetration of highly cationic proteins in vitro was found to be 240 kDa to 440 kDa. The importance of the pI for cartilage penetration is pointed out by the following examples. Three different versions of IgG antibodies (150 kDa) with different pI values were tested. Whereas an engineered IgG variant with a pI of 9.0 penetrated deeply into the cartilage, for natural IgG's having pI values of 7.0-8.0 no penetration could be detected; a third variant with a pI of 4.5 was not tested anymore. For BSA (67 kDa), a pI 8.5-9.0 variant resulted in deep penetration of the cartilage, a 7.0-8.0 variant was retained on the cartilage surface and the natural 4.5 pI variant did not reveal any detectable signal (van Lent, P. L. E. M. et al. (1987), J. Rheumatol. 14(4), pp. 798-805).

### DISCLOSURE OF THE INVENTION

Hence, it is object of the present invention to provide pharmaceutical agents against the degradation of cartilage, in particular for the treatment of osteoarthritis.

The present invention provides antigen-binding polypeptides for the treatment, prevention and/or delay of progression of cartilage degeneration and thus any disorder related thereto wherein said polypeptide is able to penetrate into the cartilage.

It has surprisingly been found that specific antigen-binding polypeptides, in particular single-chain antibodies, are able to penetrate in an effective manner into cartilage, where they can bind target proteins, such as cytokines, cytokine receptors or metalloproteinases, within the cartilage matrix. Upon binding of target molecules, their biological function can be blocked at their site of generation and cartilage degeneration can be decreased and/or inhibited. Hence, the polypeptides of the present invention are able to act in a direct manner on the specific target molecule. By adding positive charges to the polypeptide, the retention time within the cartilage can be enhanced, thereby allowing a longer contact with the target proteins.

Further, as bigger molecules cannot penetrate the cartilage matrix, a bigger volume of distribution is available upon administration of the pharmaceutical agents of the present invention when compared to bigger protein molecules.

The present invention also provides the use of said antigen-binding polypeptide for the treatment, prevention and/or delay of progression of cartilage degeneration, in particular of osteoarthritis.

The antigen-binding polypeptide disclosed herein may also be used in in vitro diagnostics and/or in vivo diagnostics of cartilage degeneration.

Furthermore, the invention encompasses a composition comprising the antigen-binding polypeptide disclosed herein and the use of said composition for the treatment, prevention and/or delay of progression of cartilage degeneration and any disorder related thereto, in particular of osteoarthritis.

In still another embodiment, a method for the treatment of cartilage degeneration is provided, wherein the antigen-binding polypeptide is locally administered, in particular by intra-articular administration.

### BRIEF DESCRIPTION OF THE DRAWINGS

The invention will be better understood and objects other than those set forth above will become apparent when consideration is given to the following detailed description thereof. Such description makes reference to the annexed drawings, wherein:

FIG. 1: Schematic drawing of the experimental set up for the in vitro cartilage penetration experiment. The following components are depicted: Pump (1), tube system, arrows indicating flow direction (2), buffer reservoir (3), diffusion chamber with: reservoir containing FITC-labeled probe (4) and flow through chamber (5), cartilage with articular surface up (towards probe reservoir) clamped to penetration chamber (6). The large arrow indicates the penetration of FITC-labeled molecules into and through the cartilage.

FIG. 2: FITC-labeled proteins that were used for cartilage penetration were diluted (1:2, 1:4, 1:8, 1:16) and spotted on glass slides to determine signal intensities under UV.

FIG. 3: The penetration of ESBA105-FITC and infliximab (Remicade®)-FITC into cartilage after the indicated time period is visualized by pictures of cartilage sections that were taken under UV light.

FIG. 4: Fluorescence intensity at a defined distance from the apical surface of bovine cartilage following incubation with FITC-labeled TNF-alpha inhibitors.

FIG. 5: Comparison of concentrations of radioactivity in the leg of male rabbits after a single i.v. (A) or single i.a. (B) administration of [125I]-ESBA105 at a dose level of approximately 1000 mcg/animal. Note, for articular cartilage following i.a. dosing, no values within the range of quantification could be obtained for the 6 hours time point. Therefore, no continuous line was drawn in the graph for this tissue.

FIG. 6: Biodistribution to knee joint tissues following i.v. and i.a. injection of [¹²⁵I]-ESBA105. Time course of [¹²⁵I]-ESBA105 levels in plasma following i.a. (dashed line) and i.v. (solid line) injection.

FIG. 7: Rescue of L929 mouse fibroblasts from TNF-α induced apoptosis. L929 mouse fibroblasts, sensitized by presence of actinomycin D were exposed to preincubated mixtures of different concentrations of ESBA105 or infliximab with rhTNF-α (final concentration 100 pg/ml). Similar to infliximab, ESBA105 blocks the pro-apoptotic effect of TNF-α in a dose-dependent manner. Potency of ESBA105 is almost identical to infliximab, as determined by an EC50 value of 12.5 ng/ml for ESBA105 and 14.0 ng/ml for infliximab, respectively.

FIG. 8: Inhibition of i.a. ESBA105 in rat monoarthritis model: Comparison of inhibitory potential of i.a. injected ESBA105 and infliximab, respectively, on acute monoarthritis induced by i.a. injection of 10 μg rhTNF-α. Effects on joint swelling (quantified by use of caliper), synovitis (HE staining; see also B) and proteglycan loss (Toluidine blue staining; see also B) were assessed.

FIG. 9: Dose response of i.a. ESBA105 in rat monoarthritis model: In vivo dose-response of ESBA105 and inflixmab, respectively. Data on synovitis and proteoglycan loss are not shown.

FIG. 10: MMP activity at day 5 in supernatant of cartilage cultures treated with FW2.3 or 20 ug/ml or 100 ug/ml ESBA105. Treatment of human osteoarthritic cartilage explants with ESBA105 significantly reduced the activity of MMPs when compared to the isotype control. The absolute values were normalized for each patient separately by setting the control condition (FW2.3) to 100%. *p<0.05. Absolute average values of MMP activity for each culture condition are given in table row below the figure.

FIG. 11: PGE2 in pooled culture medium of all time points. Both concentrations of ESBA105 significantly reduced PGE2 levels in the supernatant of the cartilage cultures. Absolute values were normalized for each patient separately by setting the control condition (FW2.3) to 100%. *p<0.05. Absolute average values of measured PGE2 levels in each culture condition are given in table row below the figure.

### MODES FOR CARRYING OUT THE INVENTION

The present invention will be described in more detail below. It is understood that the various embodiments, preferences and ranges may be combined at will. Further, depending of the specific embodiment, selected definitions, embodiments or ranges may not apply.

In the scope of the present invention, the following definitions apply:

The term “antigen-binding polypeptide” refers to the ability of a polymer of natural amino acids or non-natural amino acids to specifically bind to an antigen. These polypeptides include any antigen-binding fragment or single-chain of a full-length antibody with sufficient binding capacity for the selected antigen. Examples of antigen-binding fragments encompassed by the present invention include Fab fragments, F(ab′)2 fragments, Fd fragments, Fv fragments; single domains or dAb fragments, isolated complementarity determining regions (CDR); a combination of two or more isolated CDRs which may optionally be joined by a synthetic linker and single-chain variable fragments (scFv). “Full-length antibodies” include chimeric antibodies, in which an antigen-binding variable domain of one origin is coupled to a constant domain of a different origin, e.g. the variable domain Fv of a murine antibody to the constant domain Fc of a human antibody. The above enumerated antibodies and antibody fragments are obtained using conventional techniques known to those with skill in the art, and the fragments are screened for utility in the same manner as are intact antibodies. The term antigen-binding polypeptide further encompasses antigen-binding polypeptides that are based on alternative scaffolds which are well-known in the art and include, but are not limited to, CTLA-4, tendamistat, fibronectin (FN3), neocarzinostatin, CBM4-2, lipocalins, T-cell receptor, Protein A domain (protein Z), Im9, designed ankyrin-repeat proteins (DARPins), designed TPR proteins, zinc finger, pVIII, avian pancreatic polypeptide, GCN4, WW domain, Src homology domain 3 (SH3), Src homology domain 2 (SH2), PDZ domains, TEM-1β-lactamase, GFP, thioredoxin, staphylococcal nuclease, PHD-finger, CI-2, BPT1 APPI, HPSTI, ecotin, LACI-D1, LDTI, MTI-II, scorpion toxins, insect defensin A peptide, EETI-II, Min-23, CBD, PBP, cytochrome b₅₆₂, Ldl receptor domain A, γ-crystallin, ubiquitin, transferring, and C-type lectin-like domain (see Binz et al. (2005 October) Nat Biotech 23(10):1257-68). In a preferred embodiment of the methods and compositions disclosed herein, the antigen-binding polypeptide is a single-chain antibody.

The antigen-binding polypeptide of the present invention may be generated using routine techniques in the field of recombinant genetics. Knowing the sequences of the polypeptides, the cDNAs encoding them can be generated by gene synthesis.

The antigen-binding polypeptide disclosed herein may be labelled, for example radioactively or with a fluorescent agent, or be chemically modified, e.g. by PEGylation.

“Cartilage degeneration” may be measured by a number of methods. In preclinical experiments with cartilage explant cultures loss of collagen and/or proteoglycan can be measured directly by weighing the explant before applying the therapy and by determining the amount of glycosaminoglycan (GAG) that was released into the medium and the GAG content that remains in the cartilage. Furthermore, expression profiling of specific cytokines, such as IL-1 and TNFaplpha may give an indication of inflammatory/catabolic processes. Collagen breakdown can be determined by measuring the collagen degradation product CTX-II that was released into the culture medium. Measurement of matrix metalloproteases (MMP) expression or activity, or prostaglandin E2 (PGE2) concentrations are indirect indicators of cartilage degeneration. CTX-II can also serve as biomarker for cartilage degeneration in humans. It can be measured in the urine by means of commercial kits (e.g. CTX-II-Urin (CartiLaps®), Human from OSTEO medical GmbH, Bünde, Germany). The current standard method to assess cartilage degeneration in humans is X-ray, however it is foreseeable that this method will be replaced by magnetic resonance imaging (MRI). MRI allows quantifying articular cartilage volume and morphology (Peterfy, C G et al. (2006) Osteoarthritis and Cartilage, Volume 14, pp A95-A111). A therapeutically effective amount of an antigen-binding polypeptide refers to an amount that is needed to treat, ameliorate or prevent the disease or conditions or to exhibit a detectable therapeutic or preventive effect.

The term “pharmaceutical formulation” refers to preparations which can be administered to a subject and retain the biological activity of the antigen-binding polypeptide to be unequivocally effective, and which contains no additional components being toxic. Pharmaceutically acceptable excipients (vehicles, additives) are those which can reasonably be administered to a subject mammal to provide an effective dose of the active ingredient employed.

In a first aspect, the present invention provides an antigen-binding polypeptide for the treatment, prevention and/or delay of progression of cartilage degeneration wherein said polypeptide is able to penetrate into the cartilage.

In a preferred embodiment, the polypeptide is a single-chain antibody.

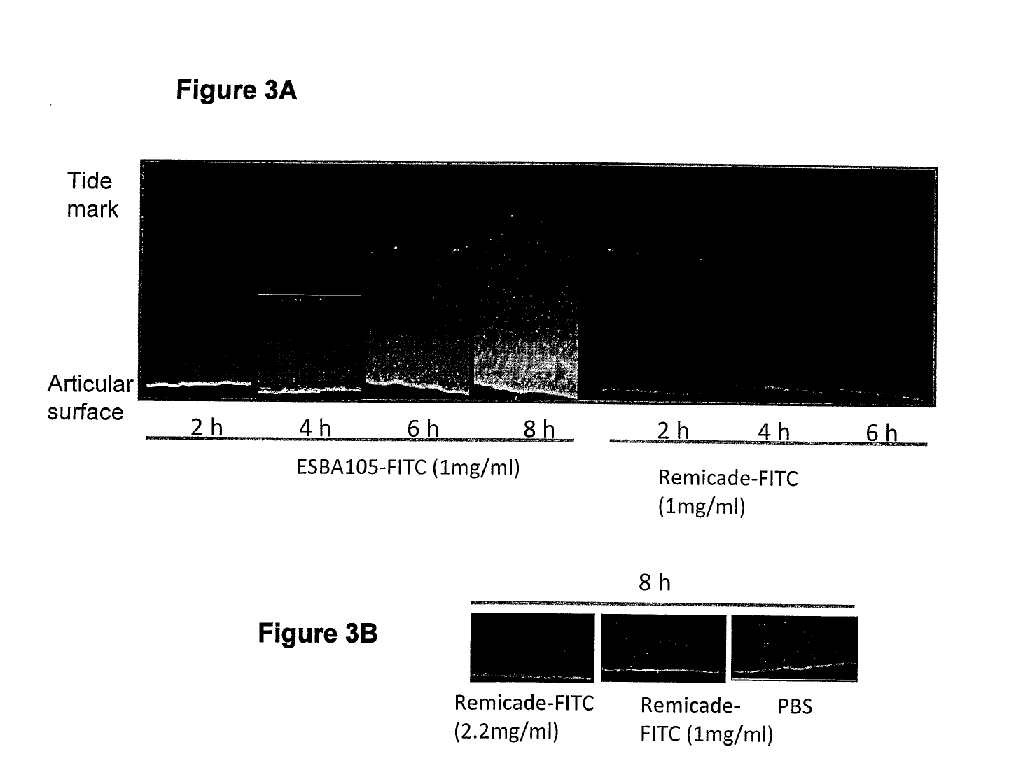

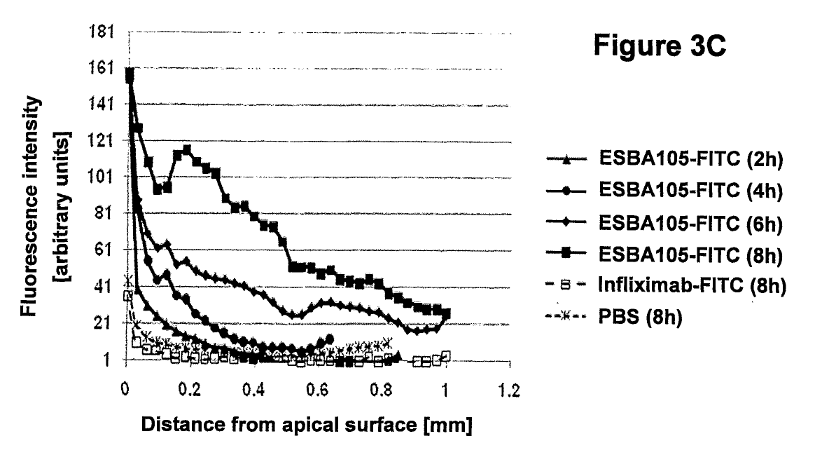

Cartilage penetration may be measured in vitro for example by applying a labeled antigen-binding polypeptide to cartilage explants, for example by the experimental set-up described in Example 1 and shown in FIG. 3. Alternatively, cartilage penetration can be assessed by radioactively labeled proteins (see e.g. van Lent, P. L. E. M. et al. (1987), J. Rheumatol. 14(4), pp. 798-805). Radioactive labelling is also suitable for determining cartilage penetration in vivo, as described in Example 2 or in van Lent, P. L. E. M. et al. (1989), J. Rheumatol. 16(10), pp. 1295-1303.

In a further preferred embodiment, the solubility of the polypeptide of the invention as measured according to the method of Atha and Ingham (1981) is at least 5 mg/ml, more preferably at least 10 mg/ml, and most preferably at least 20 mg/ml.

In particular, stable and soluble antibodies, preferably single-chain antibodies having a stable and soluble framework as described in WO 03/097697, are advantageous since highly concentrated formulations may be achieved; as a consequence thereof, small application volumes may be used. A stable and soluble antibody as referred to preferably has one or more of the following features:
it is stable under reducing conditions as measured in a yeast interaction assay, the so-called Quality control system, as disclosed in WO01/48017,
is stable for at least 1 month, preferably to at least two months, most preferred at least six months at 20° C. to 40° C., preferably at 37° C. in PBS,
it remains monomeric under physiological conditions,
it is soluble at ambient temperature in PBS at concentrations of >about 1 mg/ml, preferably of >about 4 mg/ml, more preferably of >about 10 mg/ml, even more preferably of >about 25 mg/ml and most preferably of >about 50 mg/ml,
it reveals a midpoint of transition in a guanidinium hydrochloride titration of at least 1.5 M, preferably of at least 1.75 M, more preferably of at least 1.9 M, most preferably of at least 2 M, i.e. is resistant to denaturation.

In a preferred embodiment, the polypeptide has a molecular weight of at least 10 kDa and less than 50 kDa. Preferably, the polypeptide has a molecular weight of about 26-27 kDa.

Moreover, the polypeptide preferably specifically binds a cytokine or a cytokine receptor. More preferably said cytokine or cytokine receptor is proinflammatory. Said proinflammatory cytokine is preferably TNFalpha or an interleukin, e.g. IL-1 or IL-6, or any cytokine receptor that is specific for binding of any of the listed cytokines. In another preferred embodiment, the polypeptide specifically binds a cartilage proteoglycan degrading enzyme. Such enzymes include aggrecanases and matrix metalloproteases (MMPs). Through specific binding of the target molecule, their biological activity in cartilage degeneration may be modulated and/or blocked.

In another preferred embodiment, the antigen-binding polypeptide comprises a variable light chain VL having at least 90% identity, more preferably at least 95% identity, and most preferably at least 99% identity to SEQ. ID. No. 1; and/or a variable heavy chain VH having at least 90% identity, more preferably at least 95% identity, and most preferably at least 99% identity to SEQ. ID. No. 2.

In another preferred embodiment, the polypeptide has at least 90% identity, more preferably 95% and most preferably 100% identity to sequence SEQ. ID. No. 3.

The sequences of the present invention are:

SEQ ID No: 1
VL of ESBA105
DIVMTQSPSSLSASVGDRVTLTCTASQSVSNDVVWYQQRPGKAPKLLIYS
AFNRYTGVPSRFSGRGYGTDFTLTISSLQPEDVAVYYCQQDYNSPRTFGQ
GTKLEVKR
SEQ ID No: 2
VH of ESBA105
QVQLVQSGAEVKKPGASVKVSCTASGYTFTHYGMNWVRQAPGKGLEWMGW
INTYTGEPTYADKFKDRFTFSLETSASTVYMELTSLTSDDTAVYYCARER
GDAMDYWGQGTLVTVSS
SEQ ID No: 3
ESBA105
DIVMTQSPSSLSASVGDRVTLTCTASQSVSNDVVWYQQRPGKAPKLLIYS
AFNRYTGVPSRFSGRGYGTDFTLTISSLQPEDVAVYYCQQDYNSPRTFGQ
GTKLEVKRGGGGSGGGGSGGGGSSGGGSQVQLVQSGAEVKKPGASVKVSC
TASGYTFTHYGMNWVRQAPGKGLEWMGWINTYTGEPTYADKFKDRFTFSL
ETSASTVYMELTSLTSDDTAVYYCARERGDAMDYWGQGTLVTVSS

The percent identity between two sequences is a function of the number of identical positions shared by the sequences, taking into account the number of gaps, and the length of each gap, which need to be introduced for optimal alignment of the two sequences. The comparison of sequences and determination of percent identity between two sequences can be accomplished using a mathematical algorithm, which is well known to those skilled in the art. The identities referred to herein are to be determined by using the BLAST programs (Basic Local Alignment Search Tools; see Altschul, S. F., Gish, W., Miller, W., Myers, E. W. & Lipman, D. J. (1990) “Basic local alignment search tool.” J. Mol. Biol. 215:403-410) accessible in Internet. BLAST protein searches can be performed with the XBLAST program, score=50, wordlength=3 to compare amino acid sequences to the protein molecules of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used.

In still another preferred embodiment, the penetration efficiency is dependent on the size and the pI of the antigen-binding polypeptide in relation to the pH found within the cartilage or the site of dosing. For example, the pH within a healthy knee joint is about 7.4. In an inflamed joint the pH can go down to about 7. Preferably, the pI of an antigen-binding polypeptide is higher than 7.0, more preferably higher than 7.4 and most preferably it is 7.8 or higher.

In still another preferred embodiment, the antigen-binding polypeptide is applied in a formulation providing an overall positive charge to the antigen-binding polypeptide in order to facilitate cartilage penetration and to optimize cartilage retention.

In a second aspect, the invention provides the use of the disclosed antigen-binding polypeptide for the treatment, prevention and/or delay of progression of cartilage degeneration, in particular of osteoarthritis.

The antigen-binding polypeptide disclosed herein may also be used in in vitro diagnostics and/or in vivo diagnostics of cartilage degeneration, in particular of osteoarthritis.

In still another aspect, the antigen-binding polypeptide can be used for the production of a medicament for the treatment, prevention and/or delay of progression of or as an in vitro diagnostic agent for detection of cartilage degeneration, in particular osteoarthritis.

Further, the present invention encompasses a composition comprising the antigen-binding polypeptide disclosed herein. The composition is preferably a pharmaceutical composition and may further comprise a pharmaceutically acceptable carrier or one or more further effective agents.

In a preferred embodiment, the antigen-binding polypeptide of the composition is an scFv and specifically binds TNFalpha. The antigen-binding polypeptide may be subjected to lyophilisation prior to its incorporation into the composition. Preferably, the composition is an aqueous formulation. Said aqueous formulation may be prepared dissolving the polypeptide in a pH-buffered solution, wherein the buffer has a pH above 6.0, preferably in the range from above 6.0 to 7.8. Examples of buffers that will control the pH within this range include organic acid buffers such as acetate (e.g. sodium acetate), succinate (such as sodium succinate), gluconate, histidine, and citrate.

The antibodies and compositions of the present invention can be administered to a number of different subjects, preferably warm-blooded animals, more preferably mammals, including humans and non-human animals, e.g rats, mice, rabbits, dogs, horses, cattle. In a preferred embodiment of the methods, the antigen-binding polypeptides and/or the compositions disclosed herein, the subject is a human.

The way of administration of the methods, the antigen-binding polypeptides and/or the compositions disclosed herein is preferably parenteral and most preferably intra-articular.

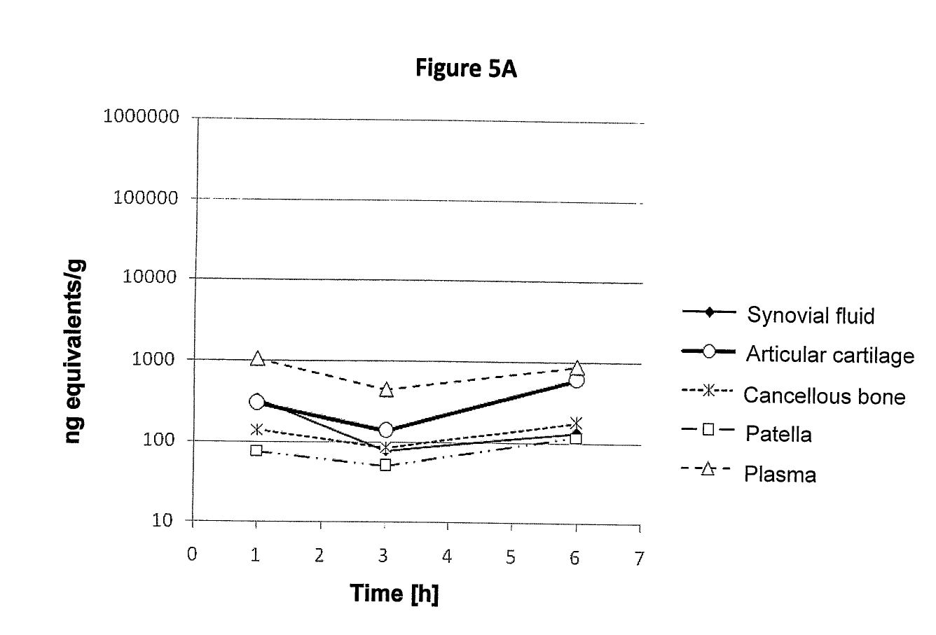

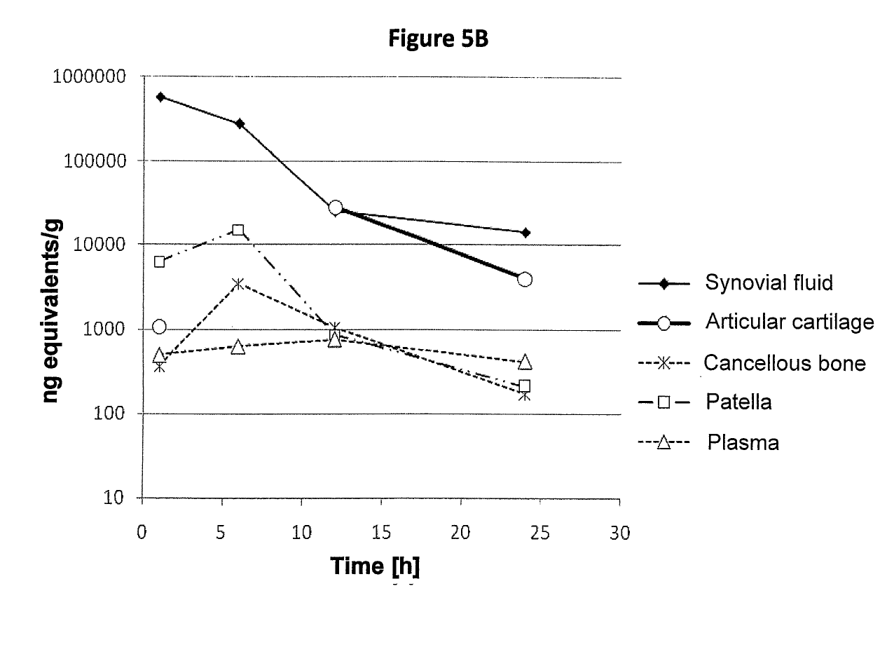

Experiments disclosed by this invention that performed in mammals (see Exp. 1) showed that after intra-articular administration of an antigen-binding polypeptide of the present invention, the polypeptide penetrates in a much more efficient manner into the cartilage (see FIGS. 5A and B) than following the intravenous route of application. In the plasma, the maximal concentration measured (Cmax) of an antigen-binding polypeptide of the present invention was lower following i.a. administration than after i.v. injection. In the particular experimental setting, the difference was 10-fold. Moreover, Cmax in the plasma peaked several hours after i.a. administration, which indicates a relatively slow absorption into the circulation from the site of i.a. injection, which is comparable to a sustained-release effect. These findings confirm the hypothesis that local administration of the antigen-binding polypeptide is preferred over systemic administration, as at the site of injection a higher local concentration of the polypeptide and a retarded clearance into the plasma is observed. Moreover, after i.a. application the systemic exposure of the polypeptide is lower when compared to intravenous administration, which reduces potential systemic adverse reactions.

Preferably, the polypeptide and/or compositions disclosed herein are chosen such that upon i.a. administration, the peak concentration Cmax of the polypeptide in the plasma is about 10fold lower then after i.v. injection, preferably more than 10fold lower then after i.v. injection. Further, the polypeptide and/or composition is chosen such that in articular cartilage, the peak concentration Cmax of said polypeptide upon i.a. administration is preferably at least 40fold higher that then after i.v. injection, preferably at least 45fold higher that then after i.v. injection. Preferably, the polypeptide exposure in articular cartilage based on AUC0-6 is about 135 fold higher and/or the AUC0-240 is 150- to 500-fold higher with i.a. as compared to i.v. application of the polypeptide or compositions disclosed herein. As mentioned above, the antigen-binding polypeptide is preferably chosen such that it has a pI higher than 7.0, and/or the composition is chosen to have a formulation which provides an overall positive charge to the antigen-binding polypeptide.

Compositions intended for parenteral and/or intra-articular use may be prepared according to any method known to the art for the manufacture of pharmaceutical compositions and may contain, besides the effective substance of the present invention, one or more agents, such as preserving agents and/or adjuvants. The composition may contain the active ingredient in admixture with suitable physiologically acceptable excipients. Such excipients include, for example, inert diluents (e.g., calcium carbonate, sodium carbonate, lactose, calcium phosphate or sodium phosphate), granulating and disintegrating agents (e.g., corn starch or alginic acid), binding agents (e.g., starch, gelatin or acacia) and lubricating agents (e.g., magnesium stearate, stearic acid or talc).

In a preferred embodiment, the composition comprises a polypeptide having analgesic and/or anti-inflammatory properties. This is in particular the case when the polypeptide specifically binds TNFalpha. In a further preferred embodiment, the composition further comprises an analgesic and/or non-steroidal anti-inflammatory drug other than the polypeptide described herein.

In another preferred embodiment, the composition comprises hyaluronic acid and/or intraarticular injected glucocorticoids.

The compositions disclosed herein are preferably formulated in a stable manner. A stable formulation is one in which the antigen-binding polypeptide therein essentially retains its biological activity, and preferably its physical stability and/or chemical stability upon storage. Various analytical techniques for measuring protein stability are available in the art and are reviewed in Peptide and Protein Drug Delivery, 247-301, Vincent Lee Ed., Marcel Dekker, Inc., New York, N.Y., Pubs. (1991) and Jones, A. Adv. Drug Delivery Rev. 10: 29-90 (1993), for example. Stability can be measured at a selected temperature for a selected time period. Preferably, the formulation is stable at room temperature (about 25° C.) or at 40° C. for at least 1 month and/or stable at about 2-8° C. for at least 1 year, preferably for at least 2 years. Furthermore, the formulation is preferably stable following freezing (to, e.g., −70° C.) and thawing of the formulation.

Since intra-articular injections are demanding invasive procedures, it is not recommended to administer them on a too frequent basis. It is therefore preferred that a pharmaceutically active compound shows a high residence time at the site of injection and/or in the joint tissues. As the polypeptide described herein is able to penetrate cartilage, upon i.a. administration, the release of said polypeptide from the joint occurs over an extended period of time. In a preferred embodiment, a prolonged residence time is further achieved by formulating the pharmaceutical composition disclosed herein as a sustained-release composition, i.e., a formulation which allows for a prolonged release, preferably on a zero order rate, of the effective compound following administration.

Such formulations may generally be prepared as fluid aqueous colloidal suspension using well known technology. The formulation is preferably sufficiently fluid to be easily injectable. Furthermore, the formulation is preferably stable in liquid form, biocompatible and biodegradable, non-toxic, non-immunogenic and has an excellent local tolerance. Sustained release formulations preferably provide a relatively constant level of modulator release. Several approaches are known in the art. In one approach, the formulation comprises at least one polymer and one active agent which are liquid and injectable and become more viscous after administration to the subject, due to a change in pH and/or temperature. Another alternative is the formation of a gelled deposit. Upon administration, the fluid gels because the temperature of the subject is above the gelling point of the gelling agent. Still another approach consists in incorporating the active agent into microspheres or implants which are subsequently administered to the subject. A forth approach is the loading of nanoparticles with the antigen-binding polypeptide. The particles are then administered as low-viscosity liquid suspensions.

The polypeptides and/or compositions disclosed herein can be used e.g. for the treatment, prevention and/or delay of progression of cartilage degeneration and any disorder related thereto. Preferably, said disorder is osteoarthritis. Within the scope of the present invention, said disorders related to cartilage degeneration encompass rheumatoid arthritis, ankylosing spondylitis, psoriatic arthritis and juvenile idiopathic arthritis, among others.

Preferably, a therapeutically effective amount of the polypeptide and/or the composition disclosed herein is administered to a subject in need thereof. The appropriate dosage is dependent on a multiplicity of factors such as the condition to be treated, the severity and course of the condition, whether the antibody is administered for preventive or therapeutic purposes, previous therapy, the patient's clinical history and response to the antigen-binding polypeptide, the type of antigen-binding polypeptide used, and the discretion of the attending physician.

The invention further encompasses an article of manufacture comprising one or more containers holding the composition. Suitable containers include, for example, bottles, vials and syringes, which may be formed from a variety of materials such as glass or plastic. The article of manufacture may further include other materials desirable from a commercial and user standpoint, including buffers, diluents, filters, needles, syringes, and package inserts with instructions for use. Further, the invention encompasses a DNA sequence which encodes the antigen-binding polypeptide disclosed herein.

In another aspect, the invention also encompasses a cloning or expression vector containing said DNA sequence.

The invention further discloses a suitable host cell transformed with said expression vector. Said host cell may be a prokaryotic host cells, preferably E. coli, or a eukaryotic host cell, such as yeast, preferably S. cerevisiae, insect cells, mammalian cells or plant cells. Preferably, said method provides an scFv antibody purified from E. coli inclusion bodies or from the E. coli periplasm, if the scFv construct used comprises a signal sequence that directs the polypeptide to the periplasm.

In still another aspect, the invention encompasses a method for the treatment prevention and/or delay of progression of cartilage degeneration comprising the steps of

(a) providing a antigen-binding polypeptide having a molecular weight of at least 10 kDa and less then 50 kDa and further having a pI higher than 7,0; and

(b) locally administering said polypeptide to a subject in need thereof.

Said polypeptide is preferably the polypeptide disclosed herein. In particular said polypeptide preferably binds to a cytokine or a cytokine receptor, preferably TNFalpha or an interleukin. More preferably, said polypeptide is stable and soluble. Most preferably, said polypeptide has at least 90% identity, more preferably 95% and most preferably 100% identity to sequence SEQ. ID. No. 3.

Optionally, the polypeptide was engineered to increase the positive charge of said polypeptide and/or said polypeptide is applied in a composition having a formulation which provides an overall positive charge to the antigen-binding polypeptide. The positive charge of the polypeptide can e.g. be increased by genetic engineering, e.g. by substitution of one or more amino acids and/or by chemical modification of the polypeptide. Thereby, cartilage penetration may be facilitated and/or cartilage retention may be enhanced.

The way of administration is preferably parenteral administration, more preferably intraarticular administration.

Typically, a therapeutically effective amount of the polypeptide is administered to the subject in need thereof. Said subject is preferably a mammal, more preferably a human being.

### Example 1

### Size-Dependent Penetration of FITC-Labeled TNFalpha Antagonists into Bovine Cartilage

The aim of the experiment was to compare the ex-vivo cartilage penetration of the anti-TNFalpha single-chain antibody (scFv) ESBA105 (sometimes also referred to as E105 in the figures) with the full-length anti-TNFalpha antibody infliximab.

Materials and Methods

ESBA105 was produced as described in WO08/006,235. Infliximab/Remicade® was purchased in an official Swiss pharmacy.

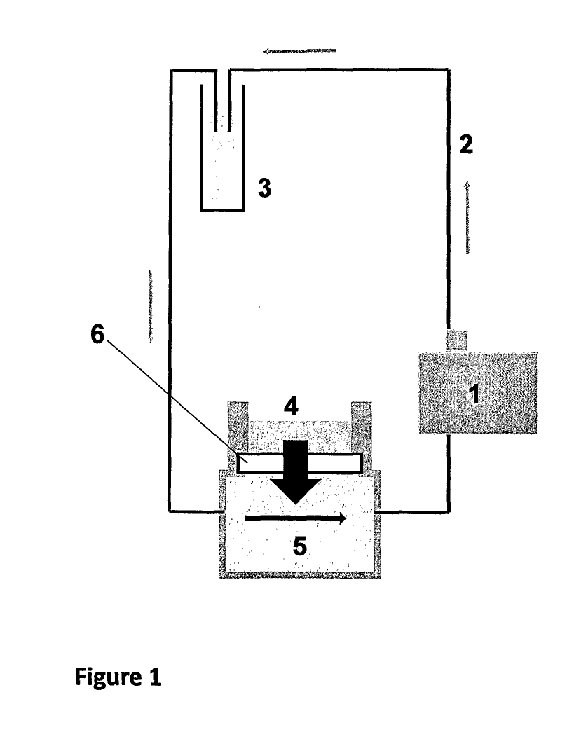

Cartilage preparations were dissected from bovine femur (freshly obtained from a slaughterhouse) and mounted in a corneal perfusion chamber as schematically depicted in FIG. 1). The cartilage layer that is naturally exposed to the synovial liquid was exposed to 300 mcl of FITC-labeled antibody solution. The tested concentrations of FITC-labeled antibodies in PBS buffer pH 7.4 were 1 mg/ml for E105-FITC and 1 mg/ml and 2.2 mg/ml, respectively, for Infliximab-FITC. The total fluid volume that circulated through the chamber, the tubing and the reservoir was 5 ml. After the designated incubation time (2, 4, 6 or 8 hours), the cartilage tissue was washed three times with 20 ml PBS pH 7.4 and subsequently embedded in OCT compound (TissueTek) and frozen in liquid nitrogen. The sample was wrapped in paraffin film (Parafilm) and stored until sectioning at −20° C.

Sectioning was performed at a section-thickness of 14 mcm using a MICROM cryostat (OT: −18° C., Knife: −20° C.). Mounted sections were analyzed and photographed under UV-Microscope (Leica) at a magnification of 40-100×. Signal intensities on photographs were analyzed using IMAGE QUANT (5.0) software.

FITC-labeling was carried out as follows: 75 mcl of freshly prepared 1 mg/ml NHS-FITC/DMSO solution were added to 1 ml of 2 mg/ml antibody solution while vortexing and incubated at room temperature for 45 minutes. The separation of unbound FITC from labeled proteins was performed by dialysis using 5 ml dialysis-cassettes in 5 l PBS pH 6.5 at 4° C. Dialysis was performed over 48 hrs, during which the dialysis buffer was completely replaced four times.

The cartilage preparations had different thickness due to excision with scalpel from bone. The cartilage surface that was exposed to the formulation is oriented towards the bottom of each photograph. Photographs 3 to 5 of figure “in vitro cartilage pentration” (from left to right) are composed of two (photograph 4 of three) photographs taken subsequently and overlayed to produce an overview of the whole examined cartilage tissue.

Results

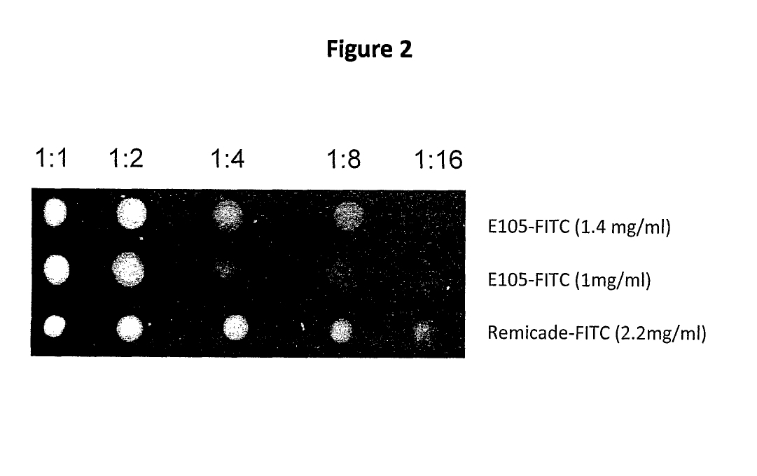

The results of the penetration study of ESBA105-FITC and infliximab-FITC into bovine cartilage are shown in FIG. 3. The time course studies reveal that ESBA105-FITC efficiently penetrates in a time-dependent manner into bovine cartilage, whereas Infliximab-FITC does not. For Infliximab-FITC there is no time-dependent penetration observed and even after 8 hours and at a concentration of 2.2 mg/ml, the picture is indistinguishable from the PBS treated cartilage. In order to allow a comparison of the different labeled protein preparations, before using them for the cartilage penetration experiment, aliquots thereof were diluted (1:2, 1:4, 1:8, 1:16) and spotted on glass slides to determine signal intensities under UV. The result of this dilution series is depicted in FIG. 2, which shows that the FITC-labeling worked equally efficient for both proteins and therefore the results of the penetration experiments are directly comparable on a qualitative basis.

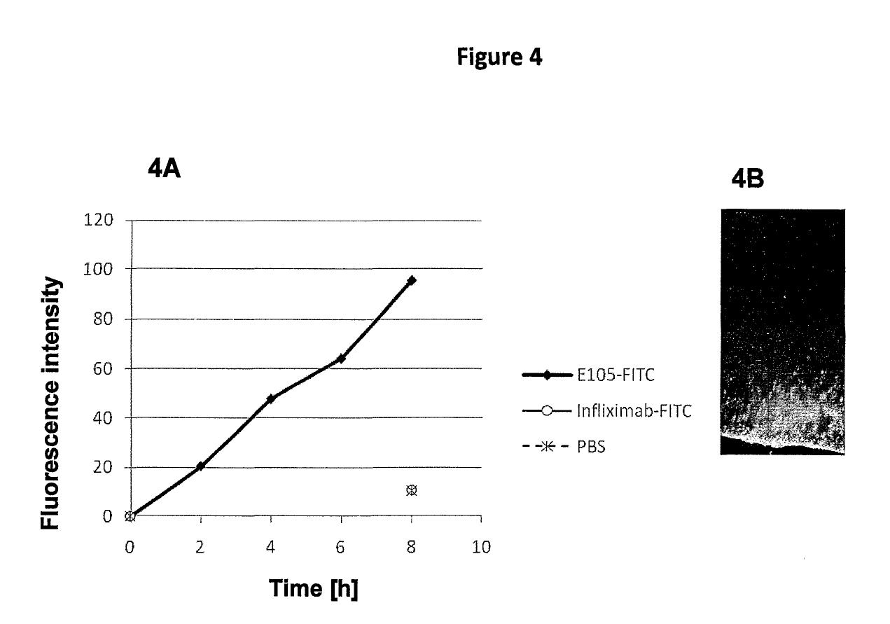

FIG. 4 A depicts a quantification of the signal intensities measured at distance 0.5 [arbitrary unit] (see FIG. 4 B for ruler) from the apex during the time course studies. For PBS and for infliximab-FITC, the measured values were almost identical and indicate that no Infliximab-FITC penetrated into the cartilage. For ESBA105, the quantitative analysis revealed an almost linear increase in signal intensity over time.

### Example 2

### In Vivo and Biodistribution Studies

Materials and Methods

TNF-α inhibitors. ESBA105 was expressed in to and purified from E. coli as described and used in 25 mM sodium phosphate pH 6.5. Infliximab (Remicade®) and etanercept (Enbrel®) were purchased in a local pharmacy.

TNF-α induced apoptosis in cell culture. Mouse L929 fibroblasts between passages pₓ6 and pₓ15 were seeded in 96-well plates (167008, Nunc, Langenselbold, Germany) in 100 μl assay medium (phenol red-free RPMI with L-Glutamine+5% FCS) to a cell density of 20,000 cells/well. Cells were incubated overnight at 37° C. and 5% CO₂. On the following day agonist-inhibitor mixtures containing recombinant human rhTNF-α (300-01A, PeproTech, London, UK) and varying amounts of ESBA105 or infliximab were prepared and incubated for 30 minutes at ambient temperature. Fifty μl of agonist-inhibitor mixtures (final rhTNF-α concentration 100 pg/ml) were given to cells subsequent to the addition of 50 μl of actinomycin D (final concentration 1 μg/ml) to each well. Cells were incubated for 20 hours. Then, 50 μl of a solution containing 1 mg/ml XTT in phenol red free RPMI and 25 μM PMS (P9625, Sigma-Aldrich, Buchs, Switzerland) was added to cell cultures and cells were incubated for another 90 minutes at 37° C. Proliferating cells express the mitochondrial succinate-tetrazolium reductase system, which metabolizes the tetrazolium salt XTT into a red product. Red color intensity was assessed by measuring absorption at 450 nm in a plate reader (TECAN, Genios, Switzerland).

Monoarthritis model. ESBA105, infliximab, or an scFv consisting of the same variable domain framework as ESBA105 but with irrelevant specificity (named here “naïve” scFv; ESBATech) each injected in 40 μl PBS followed 5 min later by rhTNF-α in 10 μl PBS were injected intraarticularly through the infrapatellar ligament of the knee of female 10 weeks old Lewis rats (Jackson) using a 28-gauge needle according to Bolon et al. 2004. For this, rats were anaesthetized with 50 mg/kg ketamine. Rats were monitored before and during the study and knee diameters were measured with calipers (Dyer, Lancaster, Pa.) pre-study and at 48 hours following rhTNF-α injection. For histopathological evaluation (Bolon B, Campagnuolo G, Zhu L, Duryea D, Zack D, Feige U. Interleukin-1beta and tumor necrosis factor-alpha produce distinct, time-dependent patterns of acute arthritis in the rat knee. Vet Pathol 2004; 41:235-243) rats were euthanized at 48 hours. Decalcified knee sections were evaluated following HE or toluidine blue staining. Sections were scored for inflammation (0 to 4), and cartilage (0 to 4) as described before (Bolon. B et al (2004), see above). Ethical approval has been obtained for all animal procedures.

Biodistribution studies. ESBA105 was labeled with ¹²⁵Iodine (¹²⁵I) to a starting specific activity of 18.6 MBq/mg using the Chloramin T method by MDS Pharma Services Switzerland AG (Fehraltorf, Switzerland).

Biodistribution studies were performed at Covance Laboratories Ltd. (Harrogate, UK) and were conducted in compliance with the United Kingdom (GLP Monitoring Authority, Medicines and Healthcare products Regulatory Agency (MHRA)) Good Laboratory Practice Regulations 1999, Statutory Instrument 1999 No. 3106 as amended by the Good Laboratory Practice (Codification Amendments Etc.) and were approved by the local Ethics Committee. Male New Zealand White rabbits received a single i.v. or i.a. dose of [¹²⁵I]-ESBA105 at a target ESBA105 dose level of 1000 μg/animal. The administered doses were in the range of 884 to 1034 μg/animal, equivalent to radioactive doses of between 0.707 and 0.827 MBq. After dosing, samples from animals were taken as described in Table 1.

TABLE 1

| Dose | Dose | | Number/Sex |
|---|---|---|---|
| group | route | Samples and sampling time | of animals |
| A | i.v. | Serial plasma samples for | 1 male |
| | | gamma-counting at 2, 10 and | |
| | | 30 minutes and 1, 3, 6, 12 | |
| | | and 24 hours post-dose | |
| | | (single animal) | |
| B | i.v. | Terminal plasma and tissue | 3 males |
| | | samples for gamma-counting | |
| | | and the right knee joint for | |
| | | cryo-sectioning and | |
| | | autoradiography evaluation at | |
| | | 1, 3 and 6 hours post-dose | |
| | | (single animal/sampling | |
| | | time). | |
| C | i.a. | Serial plasma samples for | 4 males |
| | | gamma-counting until terminal | |
| | | time point (as appropriate). | |
| | | Sampling times (for plasma): | |
| | | 10 minutes, 30 minutes and 1, | |
| | | 3, 6, 12 and 24 hours. The | |
| | | treated knee joint was taken | |
| | | for cryo-sectioning and | |
| | | autoradiography evaluation at | |
| | | 1, 6, 12 and 24 hours post- | |
| | | dose (single animal/sampling | |
| | | time). | |

Blood samples were centrifuged to prepare plasma, which was subjected to gamma counting (Packard Cobra 2 gamma counters, Perkin Elmer Life Sciences, Waltham, Mass.) to determine radioactivity concentrations for pharmacokinetics (group A). For groups B and C, animals were sacrificed by pentobarbitone overdose followed by exsanguination. The right hind leg from each animal (including the knee joint) was removed and immersed in a mixture of hexane and solid carbon dioxide for at least 15 minutes. Once fully frozen, the leg was embedded in a mould containing frozen 2% (w/v) aqueous carboxymethyl cellulose paste. The block was mounted onto the stage of a Leica CM3600 cryomicrotome maintained at about −20° C. (Leica Microsystems, Bucks, UK) and sagittal sections (nominal thickness 30 μm) were obtained through the knee joint. The sections, mounted on “Invisible-Tape” (Supapak, Shipley, UK), were freeze-dried in a GVD03 bench-top freeze drier (Girovac Ltd., Norwich, UK) and placed in contact with FUJI imaging plates (type BAS MS, Raytek Scientific Ltd, Sheffield, UK). ¹²⁵I-blood standards of appropriate activity (also sectioned at a nominal thickness of 30 μm) were placed in contact with all imaging plates. After exposure in a copper-lined, lead exposure box for 14 days, the imaging plates were processed using a FUJI FLA-5000 radiography system (Raytek Scientific Ltd). Electronic images were analysed using a PC-based image analysis package (Seescan Densitometry software, LabLogic Ltd, Sheffield, UK). The ¹²⁵I standards included with each autoradioradiogram were used to construct calibration lines over a range of radioactivity concentrations. Approximately 2 months after dosing (equivalent to about one half-life for ¹²⁵I decay), a number of sections as well as the corresponding standards were re-exposed for 4 days to allow quantification of high levels of radioactivity. In addition, tissues taken from the residual carcass from group B were macerated and/or homogenized, prior to portions being subjected to gamma-counting.

Pharmacokinetic parameters were calculated using WinNonLin Professional software (Version 4.0.1, Pharsight Corporation, Mountain View, Calif.).

Results

Mode of action. ESBA105 blocks TNF-α ligand-receptor interaction by competitive binding to the receptor binding site of TNF-α. Data from analytical size exclusion chromatography indicate that three monomeric ESBA105 molecules bind to one TNF-α trimer (data not shown), each interacting with one of the three TNF-α monomers. ESBA105 binds to rhTNF-α with a K_{D} of 2.19×10⁻⁹ M. The binding dynamics of ESBA105 to rhTNF-α to is characterised by the rate constants kₒₙ, and k_{off} of 5.72×10⁶ M⁻¹s⁻¹ and 0.01256 s⁻¹, respectively (data not shown). Thus, the off-rate from human TNF-α is in between those of infliximab and etanercept (Scallon B et al, J Pharmacol Exp Ther 2002; 301:418-26).

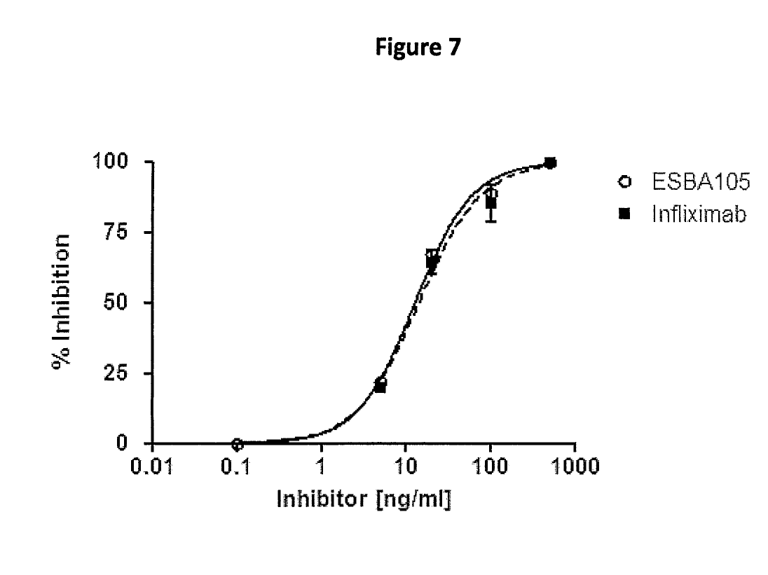

In vitro potency. The ability of ESBA105 to neutralize the biological activity of TNF-α in cell culture was demonstrated with mouse L929 fibroblasts. This cell line expresses TNF receptors I and II and upon sensitization with actinomycin D undergoes apoptosis when exposed to TNF-α. Similar to infliximab, ESBA105 in a concentration dependent manner blocked the apoptotic effect of rhTNF-α. EC50 values in the L929 TNF-α assay were 12.5 ng/ml for ESBA105 and 14.0 ng/ml for infliximab (FIG. 7).

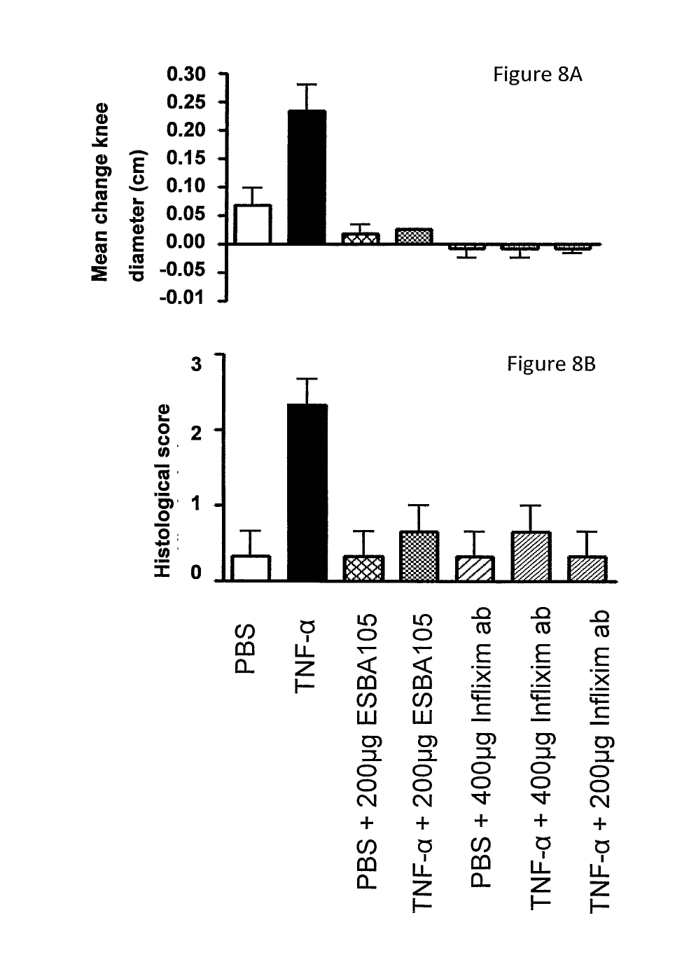

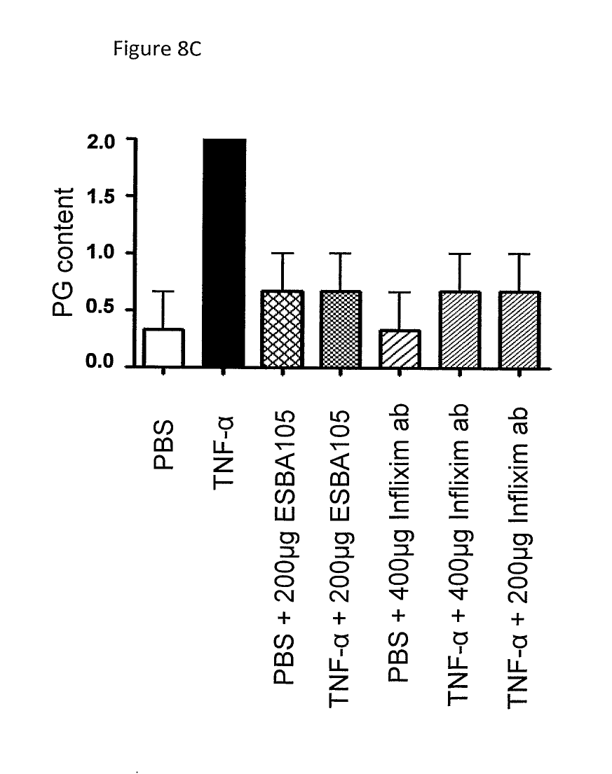

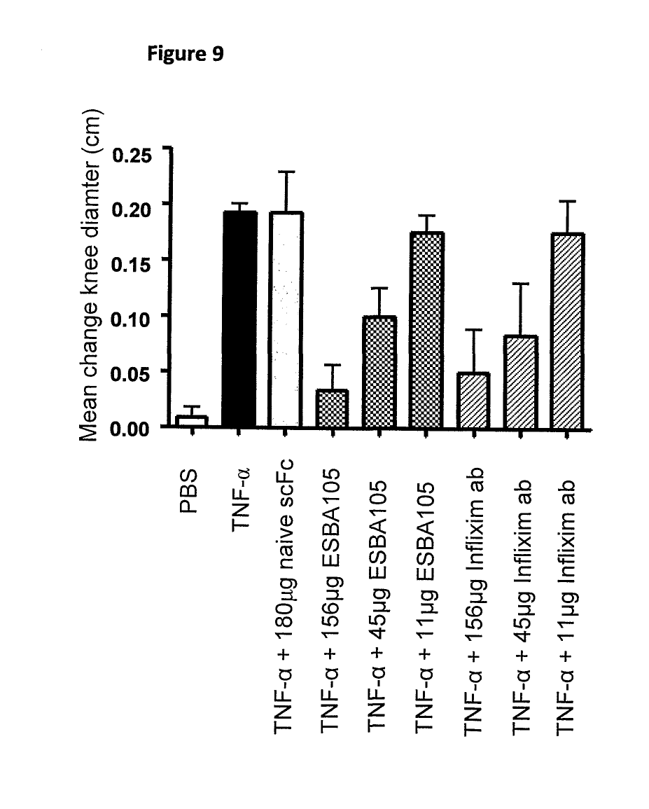

Monoarthritis model. Following i.a. injection of 10 μg rhTNF-α rat knees showed the expected inflammatory reaction (see Bolon B, Campagnuolo G, Zhu L, Duryea D, Zack D, Feige U. Interleukin-1beta and tumor necrosis factor-alpha produce distinct, time-dependent patterns of acute arthritis in the rat knee. Vet Pathol 2004; 41:235-243.): knee swelling, synovitis and loss of proteoglycan in cartilage (see rhTNF-α controls in FIG. 9). A naïve scFv with irrelevant specificity exhibited no effect on the severity of the inflammatory reactions (FIG. 9). In contrast, ESBA105 inhibited rhTNF-α caused inflammatory reactions dose-dependently (FIG. 9). Interestingly, an 11-fold molar (16-fold w/w) excess of ESBA105 over rhTNF-α resulted in 90% inhibition of knee swelling (FIG. 9). ESBA105 and infliximab demonstrated similar potency in this study (FIG. 9). Also inflammatory scores were reduced to the same extent (FIG. 8). Furthermore, proteoglycan loss in cartilage could be prevented as shown in FIG. 8.

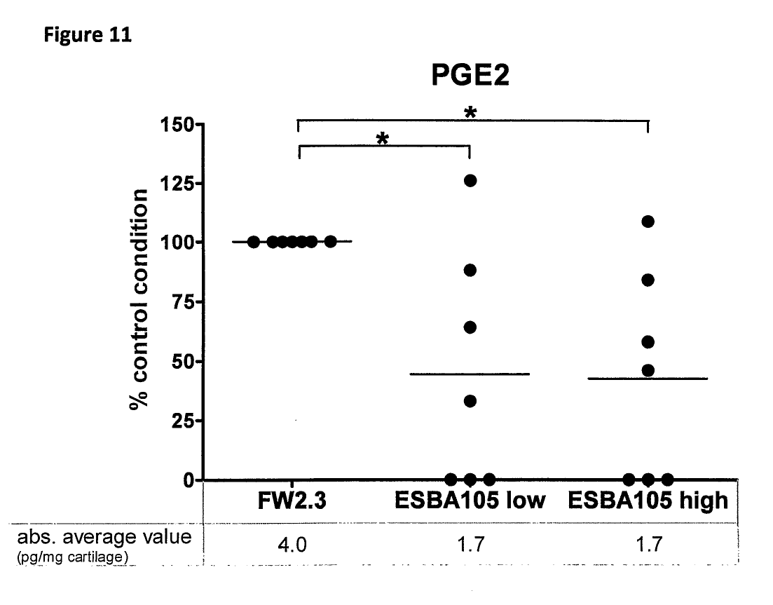

Biodistribution studies. ESBA105 is designed for local therapeutic use, in particular i.a. application to joints. First, systemic pharmacokinetics was studied comparing i.v. and i.a. application of [¹²⁵I]-ESBA105. As shown in FIG. 11A i.v. application shows the expected pharmacokinetic behavior (Larson S M, EI-Shirbiny A M, Divgi C R, Sgouros G, Finn R D, Tschmelitsch J, et al. Single chain antigen binding protein (sFv CC49)—First human studies in colorectal carcinoma metastatic to liver. Cancer Suppl 1997; 80:2458-68; Fitch J C, Rollins S, Matis L, Alford B, Aranki S, Collard C D, et al. Pharmacology and biological efficacy of recombinant, humanized, single-chain antibody C5 complement inhibitor in patients undergoing coronary artery bypass graft surgery with cardiopulmonary bypass. Circulation 1999; 100:2499-506). Measured peak concentration occurred 2 minutes post-dose (first sampling time). Thereafter, radioactivity declined in a bi-phasic manner, probably representing a distributive phase (for about 1 hour post-dose), followed by terminal elimination (Table 2).

TABLE 2
Comparison of local pharmacokinetics following local and systemic dosing

| | Tmax | | Cmax | | | T½ | | | AUC0-6 | | |
|---|---|---|---|---|---|---|---|---|---|---|---|
| | [hours] | | [ng equiv/gram] | | ratio | [hours] | | ratio | [ng equiv · h/gram] | | ratio |
| Tissue | i.a. | i.v. | i.a. | i.v. | i.a./i.v. | i.a. | i.v. | i.a./i.v. | i.a. | i.v. | i.a./i.v. |
| Plasma | 12 | 1 | 772 | 1060 | 1 | 13.5 | 19.2 | 0.7 | 15000 | 5940 | 3 |
| Articular cartilage | 12 | 6 | 28200 | 610 | 46 | 4.24 | ND | | 355000 | 2580 | 138 |
| Bone marrow | 1 | 1 | 205 | 198 | 1 | 23 | 10 | 2.3 | 3140 | 1050 | 3 |
| Cancellous bone | 1 | 6 | 3440 | 179 | 17 | 4.21 | ND | | 30600 | 941 | 31 |
| Synovial space | 1 | 1 | 574000 | 336 | 1708 | 4.13 | 3.8 | 1.1 | 3900000 | 1550 | 2516 |
| Epimysium | 6 | 1 | 249 | 218 | 1 | 14.3 | 5.81 | 2.5 | 4140 | 1080 | 4 |
| Epiphyseal line | 1 | 6 | 9320 | 338 | 28 | 4.21 | ND | | 47200 | 1740 | 27 |
| Femur | 12 | 6 | 274 | 230 | 1 | 9.02 | ND | | 3990 | 1180 | 3 |
| Muscle | ND | 1 | ND | 52 | | ND | 8.29 | | ND | 269 | |
| Patella | 6 | 6 | 15100 | 119 | 127 | 3.15 | ND | | 114000 | 565 | 202 |
| Periosteum | 24 | 6 | 438 | 343 | 1 | ND | ND | | 6100 | 1470 | 4 |
| Skin | 12 | 1 | 399 | 358 | 1 | 12.1 | 81 | 0.1 | 5870 | 2110 | 3 |
| Tibia | 12 | 6 | 223 | 308 | 1 | 14.6 | ND | | 3980 | 1250 | 3 |

i.a.—intra-articular injection
i.v.—intravenous injection
Tmax - time point of concentration peak
Cmax - peak concentration
T½ - estimated elimination half-time
AUC0-6—area under the concentration curve between zero and six hours
ND—not determined due to insufficient data

In contrast, after i.a. injection, Cₘₐₓ in plasma is reached only after 6 to 12 hours, suggesting a prolonged absorption phase. However, the AUC₀₋₂₄ in plasma is similar for both i.v. and i.a. application.

One of the advantages of local therapy is the possibility of achieving high drug levels locally. One and 24 hours after i.a. knee injection of [¹²⁵I]-ESBA105 levels of 574,000 and 14,300 ng equivalents/gram in synovial space are observed (FIG. 5B). Interestingly, ESBA105 levels in articular cartilage are almost identical in magnitude and course (FIG. 5B). In the patella, ESBA105 is absorbed slower with a Cₘₐₓ of 15,100 ng equivalents/gram at 6 hours; this level is about 20-fold lower as the levels found in synovial space and cartilage. In cancellous bone, ESBA105 following i.a. injection reaches a Cₘₐₓ of 3440 ng equivalents/gram after 6 hours (FIG. 5B). From 12 hours onwards, radioactivity diminishes in most tissues with a T½ of about 4 hours. Longer T½ are found in plasma (13.5 hours), bone marrow (23.0 hours), tibia (14.6 hours), epimysium (14.3 hours), skin (12.1 hours) and femur (9.02 hours). Interestingly, levels in synovial fluid and articular cartilage stay about 20-fold higher than in patella, cancellous bone and plasma.

In contrast to i.a. application to the knee, following i.v. application substantially lower levels of ESBA105 are found in knee joints (FIG. 11C). Exposure in articular cartilage based on AUC₀₋₂₄ is 150- to 500-fold higher with i.a. as compared to i.v. application of [¹²⁵I]-ESBA105. Terminal half lives in tissues (when they were measurable) following i.v. application were found to be comparable to those following i.a. injection (data not shown).

Discussion

Ideally, treatment of OA should address signs and symptoms as well as structure modification. However, such treatment is not available at present (for review see Goldring & Goldring, Osteoarthritis. J Cell Physiol. 2007; 213:626-34). Therefore, a pharmacological target dominantly involved in both pathophysiological processes would be ideal. TNF-α offers itself as such a target as (a) (persistent) local exposure to TNF-α causes (persistent) hyperalgesia (Sachs D et al, Pain 2002; 96:89-97; Schäfers M et al, Pain 2003; 104:579-88.), (b) TNF-α is produced by synovial tissue (Benito M J et al, Ann Rheum Dis 2005; 64:1263-7; Brennan F M et al., Scand J Immunol 1995; 42:158-65) and cartilage (Amin A R. Osteoarthrit Cartilage 1999; 7:392-4) in OA, and (c) TNF-α is a driver of inflammatory processes (Goldring S R and Goldring M B, Clin Orthop Relat Res 2004; (427 Suppl):S27-36; Schottelius A J et al, Exp Dermatol 2004; 13:193-222) and cartilage degradation (Kobayashi M et al, Arthrit Rheum 2005; 52:128-35). Furthermore, Hill et al. described a correlation of change in pain with change in synovitis during the course of knee OA (Ann Rheum Dis 2007; 66:1599-603). It has been shown that TNF-α inhibitors (a) inhibit pain and hyperalgesia (Sachs D et al, Pain 2002; 96:89-97; Elliott M J et al, Lancet 1994; 344:1105-10; Shergy W J, et al, J Rheumatol 2002; 29:667-77; Alstergren P and Kopp S, J Rheumatol 2006; 33:1734-9), (b) reduce inflammatory processes (Elliott M J et al, Lancet 1994; 344:1105-10; Feldmann M and Maini S R, Immunol Rev 2008; 223:7-19) and (c) can reverse OA cartilage from a catabolic to an anabolic state ex vivo (Kobayashi M et al., Arthrit Rheum 2005; 52:128-35).

In many patients, OA is a local phenomenon affecting a single joint such as the knee or hip (Wieland H A et al, Nat Rev Drug Discov 2005; 4: 331-344; Abramson S B and Yazici Y, Adv Drug Deliv Rev 2006; 58:212-225). Therefore, systemic TNF-α inhibition seems not appropriate due to safety considerations. Consequently, local therapy with an agent characterized by potent TNF-α inhibition, good synovial tissue and cartilage penetration, but resulting in only low systemic TNF-α inhibition would be the intervention of choice. The same argumentation holds true for treatment of mono- or oligoarthritic disease course of “classical” inflammatory arthritides (psoriatic arthritis and others). Here, we characterized the properties of such a candidate (ESBA105) for local therapy in models addressing local neutralization of TNF-α in vivo, cartilage penetration ex vivo, and biodistribution to the knee joint space of rabbits into synovial tissue and cartilage following i.a. injection in vivo. ESBA105 has a molecular weight of only 26 kDa. In contrast, currently available TNF-α inhibitors such as infliximab, etanercept and adalimumab all have a molecular weight of ˜150 kDa.

ESBA105 has nanomolar binding affinity to TNF-α and consequently inhibits TNF-α comparable to infliximab in celluar assays (FIG. 7). In vivo, in an rhTNF-α induced knee joint inflammation model in the rat ESBA105 also potently inhibits local TNF-α. In fact, an 11-fold molar (16-fold w/w) excess of ESBA105 over rhTNF-α inhibited the TNF-α induced inflammatory knee swelling, synovitis and proteoglycan loss from cartilage by 90% (FIGS. 8,9). To characterize tissue penetration capabilities of ESBA105 further, we studied penetration of ESBA105 into normal bovine articular cartilage (see Example 1). Within hours ESBA105 penetrated into cartilage. Cartilage penetration of proteins is molecular weight and charge dependent (Maroudas A, J Anat 1976; 122(Pt 2):335-47; van Lent P L et al, J Rheumatol 1987; 14:798-805; van Lent P L et al, J Rheumatol 1989; 16:1295-303). From the results it is apparent that ESBA105 has the appropriate size (26 kDa) and charge for therapeutic intra-articular use. It is noteworthy, that cartilage penetration of ESBA105 is linear (FIG. 4). Hitherto existing data support the suggestion that ESBA105 following i.a. injection will inhibit TNF-α in OA cartilage in vivo. This is expected to result in a reversal of catabolism to anabolism at least in a portion of patients according to current understanding of metabolism in osteoarthritic cartilage (compare Kobayashi M et al, Arthrit Rheum 2005; 52:128-35)). In contrast to ESBA105, an IgG (˜150 kDa) such as infliximab is too large and cannot penetrate into cartilage (FIGS. 3,4).

Biodistribution studies with [¹²⁵I]-ESBA105 in the rabbit showed, that following i.a. injection it was distributed from the knee joint and reached significantly higher levels in all OA relevant tissues following i.a. dosing than following i.v. administration of the same dose (FIG. 11, Table 4). This was most pronounced for Cₘₐₓ in synovial fluid (1.700-fold), articular cartilage (46-fold) and patella (127-fold). Distribution of radioactivity into the tissues of the leg after i.a. administration was a protracted process, with peak levels in most occurring at 6-12 hours (FIG. 5A, 5B).

In contrast, Cₘₐₓ of ESBA105 in plasma was about 10-fold lower following injection into the knee joint than after i.v. injection (FIG. 5A). In line with this finding, Cₘₐₓ in plasma following i.a. dosing was observed between 6 to 12 hours after dosing, suggesting a relatively slow absorption to the circulation from the knee joint space (FIG. 5A). Therefore, it can be expected that systemic inhibition of TNF-α is low following i.a. injection of ESBA105. All the more so, as ESBA105 is cleared more rapidly from the circulation (T½ of 7 hours in rabbits (Furrer E et al, Invest Opthalmol Vis Sci. 2009 February, 50(2):771-8. Epub 2008 Aug. 29)) than etanercept, infliximab or adalimumab (Nestorov I. Semin Arthritis Rheum 2005; 34(5 Suppl1):12-8).

Summary and Conclusions

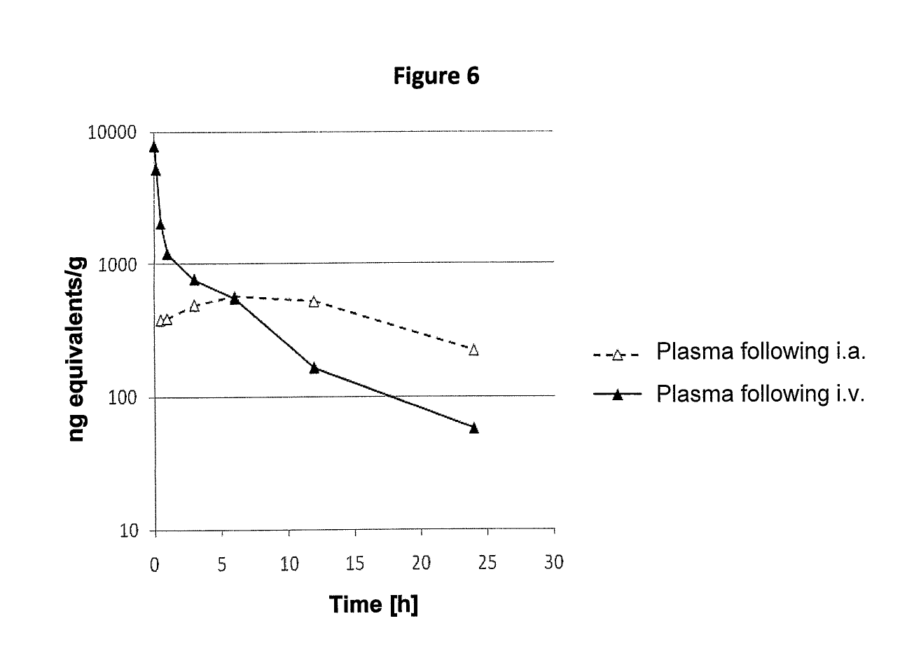

PK studies in the rabbit showed that following i.a. injection, radioactivity from [¹²⁵I]-ESBA105 was distributed into the knee joint, where it reached significantly higher levels following i.a. dosing than following i.v. administration of the same dose. This was most pronounced for synovial fluid (1′700-fold), articular cartilage (>46-fold) and patella (125-fold). See FIGS. 5 A and B for [¹²⁵I]-ESBA105distribution over time after i.v. and after i.a. administration, respectively. In vitro results confirmed efficient penetration of ESBA105 but not of infliximab into knee cartilage (see Example 1-Distribution of radioactivity into the tissues of the leg after i.a. administration was to a protracted process, with levels peaking in most tissues occurring at 6 hours (see FIG. 5B). In line with this finding, Cₘₐₓ in the plasma following i.a. dosing was observed between 6 to 12 hours suggesting a relatively slow absorption into the circulation from the knee joint space. See FIG. 6 for a comparison of the plasma concentrations of [¹²⁵I]-ESBA105 over time after i.v. and after i.a. administration, respectively. Distribution of radioactivity was widespread throughout the body after i.v. administration, with highest levels associated with the organs of elimination (kidney and the gastrointestinal tract). Taken together, these results confirm that for obtaining high and prolonged levels of ESBA105 in the knee joint and for achieving a maximized penetration into articular cartilage, the i.a. route of administration is superior over the i.v. administration.

### Example 3

### Effect of the TNF Inhibitory scFv ESBA105 on Biomarkers Related to Osteoarthritis in Human Articular Cartilage Explants from OA Patients

Methods

Study Outline and Cultivation of Human Cartilage Explants

The effect of ESBA105 on human knee articular cartilage explants from eight osteoarthritic donors on the activity of matrix metalloproteinases (MMP) and the production of PGE2 was assessed.

Human cartilage from eight different donors, suffering from knee OA, was obtained at joint replacement surgery. Groups of eight cartilage replicates from each donor were cultured in a total of three different test conditions (unspecific scFv framework of ESBA105 (FW2.3) and two different concentrations of ESBA105, see also table 3).

Full depth 3 mm diameter cartilage punches were obtained from the knee joint of patients undergoing total knee arthroplasty (TKA). The cartilage punches were weighed and brought into culture. Punches were cultured in 96-well plates each well containing one explants and 200 ul culture medium (DMEM+hydrolysed lactalbumin+50 ug/ml Vitamin C+pentomycin/streptomycin+ITS). Cartilage explants were cultured for three weeks and culture medium was refreshed twice a week. Culture medium was collected at days 5, 8, 12, 15, 19 and 21 and was stored at −80° C. until analysis.

TABLE 3
Test conditions

| culture condition | | compound | | Replicates |
|---|---|---|---|---|
| number | name | name | concentration | n |
| 1 | framework | FW2.3 | 100 ug/ml | 8 |
| | control | | | |
| 2 | ESBA105 low | ESBA105 | 20 ug/ml | 8 |
| 3 | ESBA105 high | ESBA105 | 100 ug/ml | 8 |

MMP Activity Measurements

MMP activity was measured using the fluorogenic MMP substrate TNO211-F as described in Tchetverikov et al. (Clinical and Experimental Rheumatology 2003; 21:711). This substrate is mainly converted by MMP-2, -3, -7, -9, -12 and -13. It is also converted, although at lower rate, by MMP-1. MMP activity was measured using 6.25 uM TNO211-F in the presence or absence of 5 uM BB94 (a general MMP inhibitor). Cartilage culture supernatants were diluted (final dilution 1:12) in MMP buffer and EDTA-free Complete serine and cysteine protease inhibitor was added to all samples. The difference in the initial rate of substrate conversion (linear increase in fluorescence in time) between samples with or without BB94 addition was used as a measure of MMP activity. Fluorescence was measured for 6 hours at 30° C. Test results are reported in % MMP activity of test condition compared to framework (FW) control. Statistical analysis was performed using t-test and comparing either FW2.3 with ESBA105 low or FW2.3 with ESBA105 high.

PGE2

PGE2 levels in the cartilage culture supernatants were measured using the PGE2 Assay Kit of R&D Systems (R&D Systems Europe Ltd., Abingdon, United Kingdom; cat. No. KGE004). The assay was performed according to the manufacturer's instructions using 2-fold diluted cell culture supernatants. Briefly, this assay is based on the competitive binding technique in which PGE2 present in the sample competes with a fixed amount of horseradish-peroxidase-labelled PGE2 for sites on a mouse monoclonal antibody coated onto microplates. After removing excess conjugate and unbound sample, a chromogenic substrate was added to the wells to determine bound HRP-activity. The intensity of the colour is inversely proportional to the concentration of PGE2 in the sample. Test results are reported in % PGE2 measured in the respective test condition compared to framework (FW) control. Statistical analysis was performed using t-test and comparing either FW2.3 with ESBA105 low or FW2.3 with ESBA105 high.

Results

MMP Activity

In order to determine the optimal time point for the measurement of MMP activity a test analysis was performed. Supernatants of the eight replicates at each single time point were pooled and the MMP activity in these pooled supernatants was analysed for four donors at all time points (day 5, 8, 12, 15, 19, 21) in the isotype control and the ESBA105 high culture condition. Best results were obtained at day 5. Therefore, the final analysis was performed using the supernatants of day five.

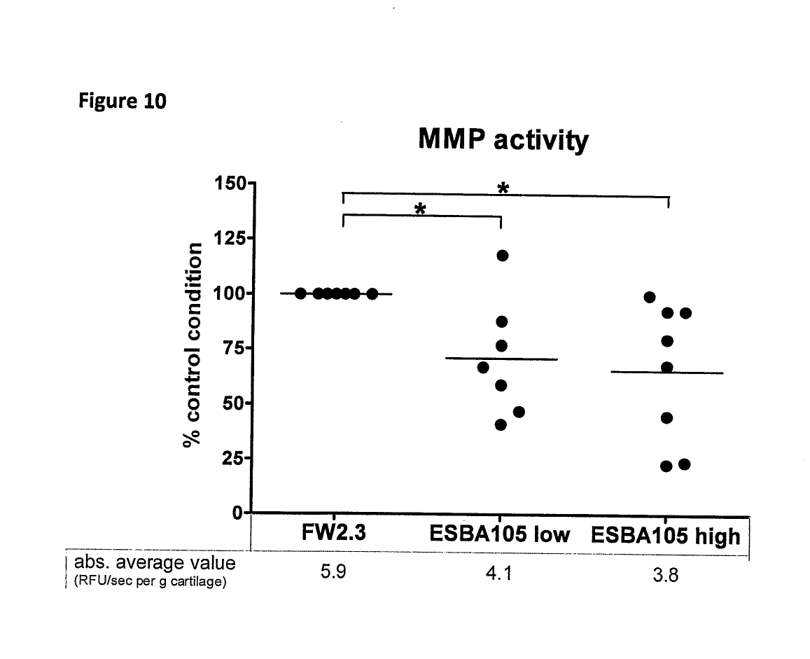

Treatment of human osteoarthritic cartilage explants with the TNF inhibitory scFv ESBA105 significantly reduced the activity of MMPs when compared to the framework control (FW2.3). Overall efficacy was similar for both concentrations of ESBA105 (FIG. 10).

PGE2

PGE2 concentrations were determined in the pooled culture medium of all replicates and time points during culture. Both concentrations of ESBA105 significantly reduced PGE2 concentrations in the supernatant of diseased cartilage cultures (FIG. 11).

While there are shown and described presently preferred embodiments of the invention, it is to be distinctly understood that the invention is not limited thereto but may be otherwise variously embodied and practiced within the scope of the following claims.

1. An antigen-binding polypeptide for the treatment, prevention and/or delay of progression of cartilage degeneration wherein said polypeptide is able to penetrate into the cartilage.

2. The antigen-binding polypeptide of claim 1, wherein the polypeptide is a single-chain antibody.

3. The antigen-binding polypeptide of claim 1, wherein the polypeptide has a solubility of at least 5 mg/ml.

4. The antigen-binding polypeptide of claim 1, wherein the polypeptide has a molecular weight of at least 10 kDa and less than 50 kDa.

5. The antigen-binding polypeptide of claim 1, wherein the polypeptide specifically binds a cytokine, a cytokine receptor or a cartilage proteoglycan degrading enzyme.

6. The antigen-binding polypeptide of claim 1, comprising a VL having at least 90% identity to SEQ. ID. No. 1; or a VH having at least 90% identity to SEQ. ID. No. 2.

7. The antigen-binding polypeptide of claim 1, wherein the polypeptide has the sequence SEQ. ID. No. 3.

8. The antigen-binding polypeptide of claim 1, wherein the pi of the antigen-binding polypeptide is higher than 7.0.

9. Use of the antigen-binding polypeptide of claim 1 for the treatment, prevention and/or delay of progression of cartilage degeneration or as an in vitro diagnostic agent for detection of cartilage degeneration.

10. The use of an antigen-binding polypeptide according to claim 9 for the treatment, prevention and/or delay of progression of or as an in vitro diagnostic agent for detection of osteoarthritis.

11. A composition comprising the antigen-binding polypeptide of claim 1.

12. The composition of claim 11, comprising an aqueous pH-buffered solution having a pH above 6.0.

13. The composition of claim 11, having a formulation suitable for intra-articular administration.

14. The composition of claim 11, having a formulation which provides an overall positive charge to said polypeptide.

15. The composition of claim 11 wherein the polypeptide is an scFv and specifically binds TNFα.

16. An article of manufacture comprising a container holding the antigen-binding polypeptide of claim 1.

17. (canceled)

18. A DNA sequence encoding the antigen-binding polypeptide of claim 1.

19. A cloning or expression vector containing the DNA sequence of claim 18.

20. A suitable host cell transformed with an expression vector according to claim 19.

21. A method for the production of an antigen-binding polypeptide comprising culturing of a host cell transformed with an expression vector containing a DNA sequence encoding the antigen binding peptide of claim 1 under conditions that allow the synthesis of said antigen-binding polypeptide and recovering it from said culture.

22. A method for the treatment prevention and/or delay of progression of cartilage degeneration wherein an antigen-binding polypeptide of claim 1 is locally administered.

23. The antigen-binding polypeptide of claim 1, wherein the polypeptide has a solubility of at least 10 mg/ml.

24. The antigen-binding polypeptide of claim 1, wherein the polypeptide has a solubility of at least 20 mg/ml.

25. The antigen-binding polypeptide of claim 5, wherein the cytokine is IL-1 or TNF alpha.

26. The antigen-binding polypeptide of claim 1, comprising a VL having at least 95% identity to SEQ. ID. No. 1; or a VH having at least 95% identity to SEQ. ID. No. 2.

27. The antigen-binding polypeptide of claim 1, comprising a VL having at least 95% identity to SEQ. ID. No. 1; and a VH having at least 95% identity to SEQ. ID. No. 2.

28. The antigen-binding polypeptide of claim 1, wherein the pi of the antigen-binding polypeptide is higher than 7.4.

29. The antigen-binding polypeptide of claim 1, wherein the pi of the antigen-binding polypeptide is higher than 7.8.

30. The composition of claim 13 which is a sustained release formulation.

31. The method of claim 22 wherein the antigen-binding polypeptide is administered by intra-articular administration.


SEQUENCE LISTING (3 sequences)

SEQ ID NO: 1 - protein, 108 aa, Artificial Sequence
  VL of ESBA105
DIVMTQSPSSLSASVGDRVTLTCTASQSVSNDVVWYQQRPGKAPKLLIYSAFNRYTGVPSRFSGRGYGTDFTLTISSLQPEDVAVYYCQQDYNSPRTFGQGTKLEVKR

SEQ ID NO: 2 - protein, 117 aa, Artificial Sequence
  VH of ESBA105
QVQLVQSGAEVKKPGASVKVSCTASGYTFTHYGMNWVRQAPGKGLEWMGWINTYTGEPTYADKFKDRFTFSLETSASTVYMELTSLTSDDTAVYYCARERGDAMDYWGQGTLVTVSS

SEQ ID NO: 3 - protein, 245 aa, Artificial Sequence
  ESBA105
DIVMTQSPSSLSASVGDRVTLTCTASQSVSNDVVWYQQRPGKAPKLLIYSAFNRYTGVPSRFSGRGYGTDFTLTISSLQPEDVAVYYCQQDYNSPRTFGQGTKLEVKRGGGGSGGGGSGGGGSSGGGSQVQLVQSGAEVKKPGASVKVSCTASGYTFTHYGMNWVRQAPGKGLEWMGWINTYTGEPTYADKFKDRFTFSLETSASTVYMELTSLTSDDTAVYYCARERGDAMDYWGQGTLVTVSS